(19) United States
(12) Patent Application Publication
(10) Pub. No.: US 2009/0227965 A1
(43) Pub. Date: Sep. 10, 2009

(54) MOTION ARTIFACTS LESS ELECTRODE FOR BIO-POTENTIAL MEASUREMENTS AND ELECTRICAL STIMULATION, AND MOTION ARTIFACTS LESS SKIN SURFACE ATTACHABLE SENSOR NODES AND CABLE SYSTEM FOR PHYSIOLOGICAL INFORMATION MEASUREMENT AND ELECTRICAL STIMULATION

(76) Inventor: RAVINDRA WIJESIRIWARDANA, Bentonville, AR (US)

Correspondence Address: RAVINDRA WIJESIRIWARDANA, 2001 LATROBE DRIVE APT 20, BENTONVILLE, AR, 72712, US

(21) Appl. No.: 12/395,768

(22) Filed: Mar. 2, 2009

Related U.S. Application Data
(60) Provisional application No. 61/033,841, filed on Mar. 5, 2008.

(51) Int. Cl. A61M 35/00; A61B 5/04; A61B 5/00; A61F 7/00

(52) U.S. Cl. 604/289; 600/372; 600/301; 607/96

(57) ABSTRACT

Motion artifacts less electrode for bio-potential measurements and electrical stimulation is discussed under the present invention. Three different arrangements of the electrode are introduced. Further the electrode embodiments are generalized for reducing motion artifacts of any skin contact senor or an actuator embodiment. In addition a piggy backed daisy chained sensor nodes or actuator nodes cabling system is introduced to minimize the motion artifacts further. A PPG sensor is constructed according to the generalized sensor embodiment and this PPG sensor is used for constructing an ear wearable heart rate monitoring unit. Moreover an ear wearable EEG monitoring system based on piggy backed daisy chained sensor or actuator nodes and caballing arrangement is illustrated.

### CROSS-REFERENCE TO RELATED APPLICATIONS

This application claims the benefit of provisional patent application Ser. No. 61/033,841, filed Mar. 5, 2008 by the present inventor.

### FEDERALLY SPONSORED RESEARCH

Not Applicable

### SEQUENCE LISTING OR PROGRAM

Not Applicable

### BACKGROUND

1. Field

This application relates to bio-potential electrodes and bio-potential electrodes caballing systems

2. Prior Art

One of the major problems of bio-potential electrodes used today is their vulnerability to the motion artifacts. This is one of the major drawbacks in patient monitoring units, rehab units, sports and health information monitoring systems. In addition when a patient or a wearer is moving the signal to noise ratio of the bio potential signals captured by these electrodes reduces due to motion artifacts. Therefore the fidelity, accuracy and reliability of the electro cardiogram (ECG), electromyogram (EMG) and electro encephalogram (EEG) signals that are measured under motion get reduced. Bio potential measuring electrodes used today adopt two methods to overcome this problem. First method is the use of large adhesive areas on the substrate of the electrode and second method is to allow some hole on the electrode substrate to clamp the lead connector wire. Both of these methods are failing under the motion since they are unable to address the issues that cause the motion artifacts. That is because the electrode's transduction zone is not isolated from the substrate of the electrode arrangement and hence the unwanted fluctuation of kinetic energy is transferred to the transduction zone of the electrode under both methods.

### SUMMARY OF THE PRESENT INVENTION

The motion artifacts of the bio potential monitoring systems mainly occur due to the relative motion of the electrode against the skin. This is further exaggerated by the bulky caballing systems and connectors that connect these electrodes to the external monitoring systems.

The present invention is a new motion artifact less bio-potential electrode and a bio potential electrodes caballing system that will reduce the motion artifacts and hence improve the signal to noise ratio.

The electrode consists of three substrates (FIG. 2A). First substrate is the electrode region part (002) and the second substrate is the external lead connector holder (005) and the third substrate holds the electrical connection path between the electrode and the connector (100). One arrangement of the electrode, the second and the third substrates are the same (FIG. 2B). In another arrangement of the electrode, there is no third substrate (FIG. 1A). The electrode region is connected to the external lead connector part by the means of an insulated electrically conductive wire, insulated electro conductive fiber/s, insulated electro conductive yarn/yarns, insulated electro conductive fabric (knitted/woven/nonwoven) or insulated electro conductive polymer.

The piggy backed daisy chained sensor nodes and caballing system shown in FIG. 3C consists of sensor nodes (FIG. 3A) to hold the sensors, the electrical connectors and electrical wires to form the signal and power pathways. There can be more than three sensor nodes, but for the explanation purposes only three are used. The first sensor (026) is connected to the next sensor substrate by electrically insulated wire or cable. There can be one or more connectors (022) on the first sensor. Also can be one or more conductive pathways or electrical cable carrying the signal from the sensor to the next sensor node receiving connector (021). On the second sensor node or the intermediate sensor node substrate there are two or more connectors (021,022). One connector (021) is for connecting the conduction pathways from the first sensor (026) and the other connector (022) is for connecting the conduction pathways carrying of the previous sensor node and it's own sensor signal and own conduction pathways (piggy back arrangement). Similarly, the rest of the sensor nodes are connected to the network to form an electrical daisy chain.

### DRAWINGS—FIGURES

FIG. 1A—Shows the detached arrangement of the electrode substrate and the lead connector substrate.

FIG. 1B—Skin contact side view of the detached substrates arrangement of the electrode.

FIG. 2A—Shows the two substrates arrangement of the electrode with electrode not in the lead connector substrate or ring substrate carrying the conduction pathways between the electrode and the lead connector.

FIG. 2B—Shows the single substrate arrangement of the electrode with the electrode not in the substrate that carry the conduction pathways between the electrode and the lead connector and the lead connector.

FIG. 3A—Shows the three dimensional view of the sensor node.

FIG. 3B—Shows the skin contact side of the sensor node.

FIG. 3C—Shows the Physiological signal monitoring system constructed with the sensor nodes.

FIG. 4A—Shows the ear wearable wireless heart rate monitoring systems with a PPG sensor constructed either by using the same physical arrangement of the FIG. 1A, FIG. 2A and FIG. 2B.

FIG. 4B—Shows an ear wearable EEG monitoring system constructed according to the piggy backed daisy chained caballing system.

FIG. 5A—ECG signal picked up from the motion artifacts less electrode arrangements.

FIG. 5B—ECG Signal picked up from the traditional sticky electrodes.

FIG. 5C—PPG signal picked up from the ear wearable PPG sensor based heart rate monitor.

FIG. 5D—EEG signal picked up from the ear wearable piggy backed daisy chained electrode nodes and caballing arrangement.

### DRAWINGS—REFERENCE NUMERALS

001—Electrode carrying substrate.

002—Electrode.

003—Wire/s carrying signals between the electrode and the lead connector.

004—Lead connector to the external signal cable.

005—Lead connector substrate.

100—Ring substrate that carries the signal pathways.

006—Conduction pathway that connects the electrode and the conduction pathway on the ring substrate (100).

007—Conduction pathway on the ring substrate.

008—Conduction path way that connects the conduction pathway on the ring substrate and the lead connector.

009—Substrate carrying the conduction path way and the lead connector that is connected to the electrode via the wire of the conduction pathway.

021—Connector for the signal pathways from the adjacent sensor nodes.

020—Sensor node substrate.

022—Connector for the signal pathways to the next adjacent sensor node.

023—Signal pathways that connects the connector (021) to the connector (022).

024—Sensors of a sensor node.

025—Intermediate sensor node.

026—First sensor node.

027—Cable carrying the signal/s or power between the sensor nodes.

028—Cable carrying the signals from the sensors nodes and power to the sensor nodes from the signal conditioning and transceiver unit.

029—Signal conditioning and transceiver unit.

030—An ear wearable PPG signal conditioning and signal transceiver unit

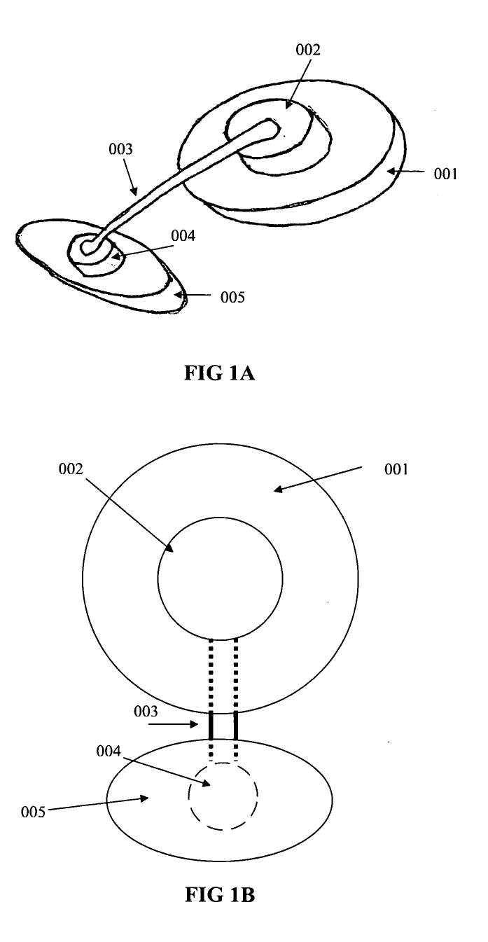

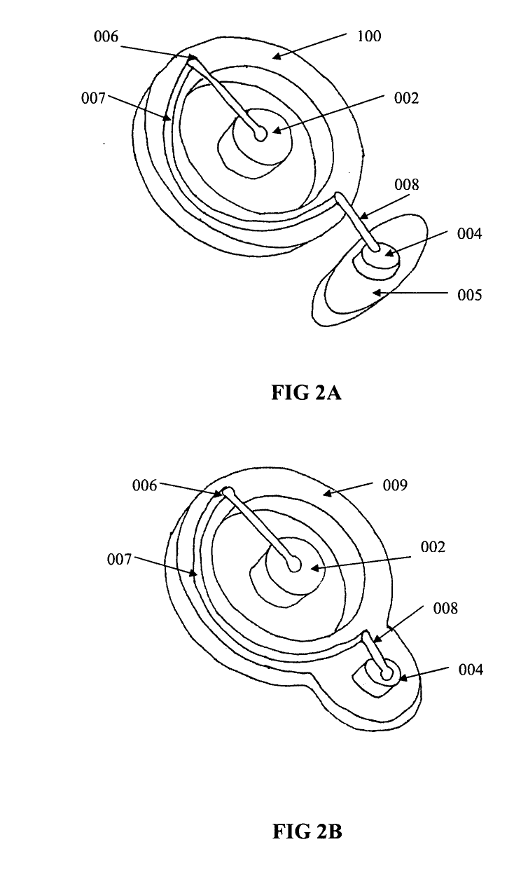

031—A pulse plethysmography (PPG/SpO₂) sensor constructed using either the same physical embodiment of the FIG. 1A, FIG. 2A or FIG. 2B.

032—Connection cable between the an ear wearable PPG signal conditioning and signal transceiver unit and the PPG sensor.

040—An ear wearable EEG signal conditioning and transceiver unit.

041—electrical caballing between the EEG sensor nodes.

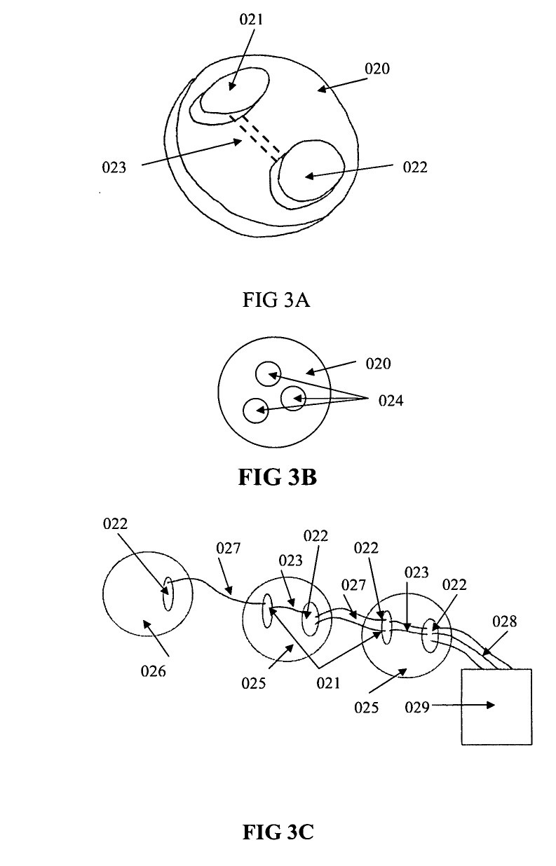

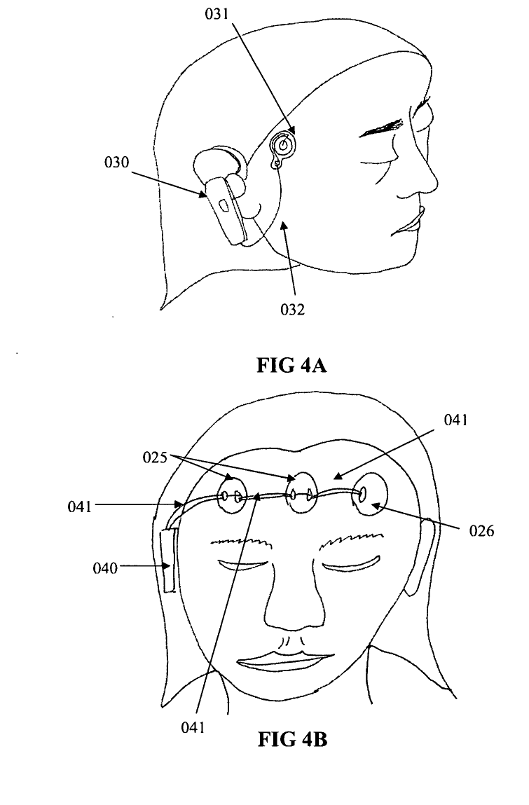

### DETAILED DESCRIPTION OF FIG. 1A, FIG. 1B, FIG. 2A, FIG. 2B, FIG. 3A, FIG. 3B, FIG. 3C, FIG. 4A, FIG. 4B

FIG. 1A and FIG. 1B show the first arrangement of the electrode. The electrode (002) is connected to a substrate (001) that can be attached on to the skin of the wearer. The electrode is connected to the lead connector (004) via the electrically conductive wire (003). The lead connector (004) is on a separate substrate (005). The external electrical cable/s is connected to this lead connector.

FIG. 2A shows the second arrangement of the electrode. The electrode (002) is not in the same substrate as the ring substrate (100). The electrode is a sticky electrode and the ring substrate is also a sticky substrate. The electrode is surrounded by the ring substrate (100). The lead connector (004) of the electrode is on a separate sticky substrate (005). Electro conductive path ways connect the lead connector (005) and the electrode (002). Part of this conductive pathway is on the ring substrate (100).

FIG. 2B shows the third arrangement of the electrode. The electrode (002) is surrounded by the extended sticky ring substrate (009) to facilitate room for the lead connector (004). The lead connector (004) connects to the electrode via the conductive pathway on the substrate (009).

FIG. 3A and FIG. 3B show a three dimensional view of a sensor node that consists of skin contact sensors (024), electrical connector to facilitate the signal pathways for the adjacent sensor nodes (021) and electrical connector to connect to the adjacent signal pathways of the adjacent sensor node (022). The two connectors are electrically connected through the conductive pathways in the substrate (023). Electrical connector 022 facilitates the signal and power pathways from the connector 021 and also the signal and power pathways from it's own sensors (024).

FIG. 3C shows a physiological information monitoring system constructed with the sensor nodes discussed in FIG. 3A and FIG. 3B. The first sensor node has the only 021 type connector and the intermediate nodes (025) contain the both type 021 and 022 connectors. The sensors (024) of a sensor node may contain bio-potential measuring electrodes, pulse plethysmography (PPG) sensors, temperature sensors, glucose sensors and any combination of them. The sensor nodes are connecting to a signal conditioning and transceiver unit (029) via a single cable comprises of multiple conductive pathways (028). The device 029 conditions the signals from the sensor nodes and supplies power to the sensor nodes. In addition it contains wireless signal communication capabilities.

FIG. 4A shows an ear wearable wireless heart rate monitoring systems with a PPG sensor constructed either by using the same physical arrangement of the FIG. 1A, FIG. 2A and FIG. 2B (031). The only difference is the electrode is replaced by a PPG sensor. The signal conditioning unit (030) is capable of wireless communication of the PPG signals. The connecting cable (032) is used for the signal and power transmission between 031 and 030.

FIG. 4B shows an ear wearable EEG monitoring system constructed according to the piggy back caballing system (FIG. 3C). The sensor nodes (026) consist of EEG electrode and three or more sensor nodes of EEG electrodes are used of the signal pickup. The signal conditioning unit (040) is capable of wireless communication of the EEG signals. The connecting cables (041) are used for the signal transmission between sensor nodes and the 040.

Operation of the Electrode, the Sensor Node and the Caballing Arrangements

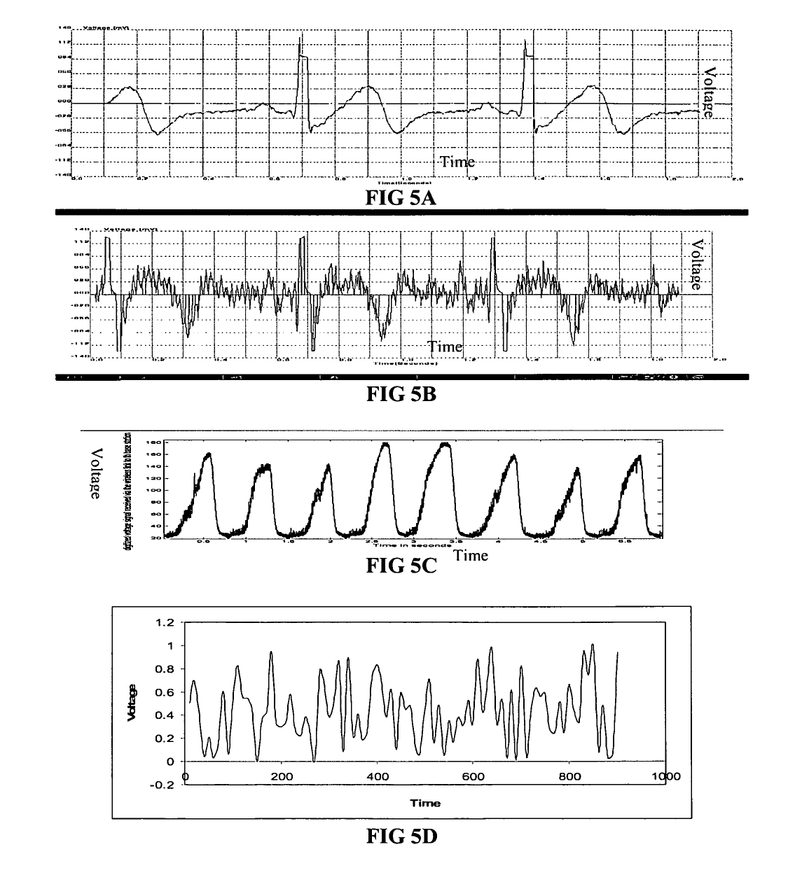

A bio potential electrode is a transducer that converts ionic responses of the physiological activities into electrical current responses. Due to construction of the electrodes discussed under FIG. 1A, FIG. 1B, FIG. 2A and FIG. 2B when a wearer is moving the unwanted mechanical energy fluctuation reaching the electrode transduction zone is minimized. This is because the lead connector is not in the same substrate as the electrode substrate and therefore the motion artifacts induced by the cable movement is minimized. In addition the ring substrate configuration illustrated under FIG. 2A and FIG. 2B further provide the stability to the electrode transduction zone hence reducing the motion artifacts and improving the signal to noise ratio. Moreover the sensor node and the caballing arrangement minimize the need to use of bulky caballing system that is very uncomfortable to wear and also reduces the weight on the electrodes hence reducing the motion artifacts and improving the signal to noise ratio. To test thee electrodes performances constructed according to FIG. 1A, FIG. 2A and FIG. 2B (new electrodes) a person wearing an ECG monitoring system with the new electrodes and a person wearing an ECG monitoring system with traditional sticky electrodes are tested under the persons running at 8-10 mph in a sweaty environment. The ECG signals of the new electrodes are shown in the FIG. 5A and the ECG signal of the traditional electrodes is shown in FIG. 5B.

It is clear that the new electrodes are capable of providing very low signal to noise ratio under most demanding conditions.

These electrode embodiments can be extended to generalized body surface attachable motion artifacts less sensor embodiments. Here the electrode (002) is replaced by the respective sensor. This sensor may be a temperature sensor, PPG sensor, glucose sensor or an ammonia sensor. FIG. 4A shows a person wearing an ear wearable PPG sensor based heart rate monitor. This PPG sensor is constructed by using the generalized motion artifacts less sensor embodiments. This device is capable of transmitting the PPG signal wirelessly to an external display unit. The PPG signal picked up from this device is shown in the FIG. 5C.

An ear wearable EEG monitoring device (FIG. 4B) is constructed with piggy backed daisy chained using the EEG electrodes nodes and caballing system (FIG. 3C). The system is capable of providing hi fidelity EEG signals. The picked up EEG signal is shown in the FIG. 5D.

I claim:

1. A bio-potential electrode having an electrode or electrodes in one attachable substrate, the electrical connector or connectors to an external cable are in another separate substrate and an electrical connection pathway or pathways between the electrodes and the connectors that connect the electrodes to the connectors.

2. Use of one or more of devices according to claim 1 in measuring ECG, EEG, EMG or skin or tissue electrical impedance.

3. A physiological information monitoring sensor having the sensor or sensors in one attachable substrate, the electrical connector or electrical connectors being in another attachable substrate that is not mechanically connected to the sensors substrate and an electrical connection between the sensors and the connectors that connect the sensors to the connectors.

4. A wearable actuator arrangement having an actuator in one attachable substrate, the connectors being in another attachable substrate and an electrical connection pathways between the actuators and the connectors that connect the actuators and the connectors.

5. A device according to claims 1 or claim 3 or claim 4 having a substrate with the connectors surrounding the substrate containing the electrode, sensors or the actuators.

6. A device according to claim 3 or claim 5 where the sensor is an PPG/SpO₂, Thermal sensor, humidity sensor, glucose sensor, blood or sweat gas sensor, inductive sensor, capacitive sensor, impedance sensor, resistive sensor, piezoelectric sensor, thermo-electrical sensor, chemical sensor, pressure sensor or an optical sensor.

7. A device according to the claim 4 where the actuator is a heater or a trans-epidermal drug delivery unit.

8. A piggy backed daisy chained sensor node and cabling system that connects skin attachable physiological information monitoring sensor nodes or actuator nodes to physiological information monitoring device or a control devices via cabling system such that.
(a) A sensor node comprises of sensors and connectors or an actuator node comprises of actuators and connectors to connect the electrical pathways from and to of an adjacent sensor or actuator nodes respectively.
(b) The connector cables are connected via adjacent substrates connectors of the attachable sensor nodes or actuator nodes and then connect to the monitoring or control device in a piggy backed daisy chained means.
(c) The sensor nodes or the actuator nodes are attachable to the skin of the person.
(d) The cables are free flowing between the sensor nodes or the actuator nodes.

9. An ECG, EMG, EEG monitoring system according to claim 8.

10. An ear wearable, a garment attachable or body attachable ECG, EMG or EEG monitoring system according to claim 8 where the sensor nodes are ECG, EMG or EEG sensors respectively.

11. A device according to claim 8 where the sensor nodes comprise of actuators or actuator nodes comprise of sensors.

12. The actuators in claim 11 are heaters, electrodes, or trans-epidermal drug delivery unit.

13. The caballing system in claim 8 comprises of electrical conduction pathways for signal and power transmission.

14. The caballing system in claim 8 comprises of hollow tubes for gas or liquid transportation.

15. An ear wearable heart rate monitoring unit with a PPG based sensors, where the PPG sensors are constructed according to claim 3.

16. A caballing systems according to claim 8 where the cables are detachable from the connectors of the nodes.

17. The nodes of the caballing system according to claim 8 are constructed with the sensors or actuator embodiment of the claim 3 or claim 4 or claim 6.
